(19) United States
(12) Patent Application Publication
(10) Pub. No.: US 2006/0159752 A1
(43) Pub. Date: Jul. 20, 2006

(54) Extended release matrix tablets

(76) Inventors: Girish Kumar Jain, Delhi (IN); Orn Anand, Delhi (IN); Ashok Rampal, Amiritsar (IN)

Correspondence Address: RANBAXY INC., 600 COLLEGE ROAD EAST, SUITE 2100, PRINCETON, NJ, 08540, US

(21) Appl. No.: 10/524,461

(22) PCT Filed: Aug. 12, 2003
(86) PCT No.: PCT/IB03/03269

§ 371 Date: Mar. 24, 2006

(30) Foreign Application Priority Data
Aug. 14, 2002 (IN) 839/DEL/2002

(51) Int. Cl. A61K 31/736; A61K 31/717; A61K 9/22

(52) U.S. Cl. 424/468; 514/54; 514/57

(57) ABSTRACT

The present invention relates to extended release matrix tablets for oral administration that include a cationic polymer, a water-swellable polymer, and an alginic acid derivative to cause the release rate of the active ingredient of the tablets to be independent of pH and gastric residence time. The active pharmaceutical ingredient may be one or more of antibiotics, sympathomimetics, sympatholytic agents, cholinergic agents, antimuscarinics, gastro-intestinal drugs, gentio-urinary smooth muscle relaxants, cardiac drugs, anticonvulsants, tranquilizers and sedatives, and in particular may be an antibiotic, such as cefaclor, or may be a sympatholytic agent, such as carvedilol.

### TECHNICAL FIELD OF THE INVENTION

The present invention relates to extended release matrix tablets for oral administration that include a cationic polymer, a water-swellable polymer, and an alginic acid derivative to cause the release rate of the active ingredient of the tablets to be independent of pH and gastric residence time.

### BACKGROUND OF THE INVENTION

Treatment of a disease or infection in most cases requires maintaining a desired drug plasma concentration level over a prolonged period of time. Such clinical needs often are satisfied by a multiple dose therapy, which can involve frequent dosing of two to four doses per day. It can be very difficult for patients to stick to such stringent routines, which can lead to poor patient compliance and, consequently, the desired drug plasma concentration level can be below the acceptable minimum therapeutic concentration. This can lead to inadequate relief and/or the development of a tolerance or resistance to the drug.

The most common approach to minimizing patient noncompliance is by using extended release drug delivery systems to decrease the number of doses that must be taken each day. One useful approach in this regard involves using a polymer-based matrix in which the drug is uniformly dispersed or dissolved. The release rate of the drug through the matrix is usually governed by the rate of dissolution of drug from the exposed surfaces and the rate of diffusion from the interior regions of the matrix to the surface.

The normal pH in the human gastrointestinal tract varies from about pH 1.0 (in fasted stomach) to about pH 8 (in lower large intestine). For drugs that have pH dependent solubility, the time of residence of the delivery system at a particular site becomes important. Such drugs can have varying release rates between the stomach and the distal regions of the intestinal tract depending on the pH at the absorption site and the gastric residence time.

Therefore, extended release matrices that can provide drug release independent of pH and gastric residence time are of particular need. One such matrix drug delivery system has been described in U.S. Pat. No. 6,150,410. This patent discloses extended release pharmaceutical compositions of acidic pharmacological agents that have reduced dependence of the release rate upon pH and gastric residence time. The extended release compositions comprise a combination of water-swellable, hydrophilic polymer and acid soluble polymer which is swellable above pH 5. These compositions provide an enhanced rate of release of the acidic pharmacological agent in the stomach where the pH of the gastric juices is low and diminished release rate at neutral or slightly alkaline pH of the intestines.

Further, U.S. Pat. Nos. 5,695,781 and 6,083,532 disclose a three component, release rate controlling matrix composition that includes a pH dependent gelling polymer such as an alginate component, an enteric polymer and a pH independent gelling polymer. Additionally, U.S. Pat. No. 6,251,430 describes the use of ethyl cellulose or Eudragit® RS or RL in combination with hydroxypropyl methylcellulose and sodium alginate to provide for a controlled release.

Despite these efforts, there remains a need for extended release pharmaceutical compositions for oral administration, from which a wide range of drugs can be released, irrespective of pH and gastric residence time.

### SUMMARY OF THE INVENTION

In one general aspect there is provided an extended release matrix tablet for oral administration which includes one or more active pharmaceutical ingredients, a water swellable cellulose derivative, an alginic acid derivative and a cationic polymer.

Embodiments of the extended release matrix tablet may include one or more of the following features. For example, the extended release matrix may be from about 10% to about 80% by weight of the total formulation.

The water swellable cellulose derivative may be one or more or hydroxypropyl methylcellulose, hydroxypropyl cellulose, methylcellulose, carboxy methylcellulose, hydroxy methylcellulose, and hydroxy ethylcellulose, and in particular may be hydroxypropyl methylcellulose and/or hydroxypropyl cellulose. The water swellable cellulose polymer may be from about 10% to about 50% by weight of the total formulation.

The alginic acid derivative may be one or more of alginic acid and its physiologically acceptable salts. The physiologically acceptable alginic acid salts may be one or more of sodium, potassium, calcium and magnesium salts of alginic acid, and in particular the physiologically acceptable alginic acid salt may be sodium alginate. The alginic acid derivative may be from about 0.1% to about 15% by weight of the total formulation.

The cationic polymer may be a methacrylic acid derivative with a dimethylaminoethyl ammonium group. The methacrylic acid derivative with a dimethylaminoethyl ammonium group may be Eudragit® E 100 and/or Eudragit® EPO. The cationic polymer may be from about 0.1% to about 15% by weight of the total formulation.

The active pharmaceutical ingredient may be one or more of antibiotics, sympathomimetics, sympatholytic agents, cholinergic agents, antimuscarinics, gastro-intestinal drugs, gentio-urinary smooth muscle relaxants, cardiac drugs, anticonvulsants, tranquilizers and sedatives, and in particular may be an antibiotic, such as cefaclor, or may be a sympatholytic agent, such as carvedilol.

The tablet may additionally contain other pharmaceutically inert excipients. The other pharmaceutically inert excipients may be one or more of binders, diluents, lubricants, glidants and colors. The binders may be one or more of methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate and propylene glycol. The diluents may be one or more of calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol sorbitol, starch, starch pregelatinized, sucrose, sugar compressible, sugar confectioners and mixtures thereof. The lubricants and glidants may be one or more of colloidal anhydrous silica, stearic acid, magnesium stearate, calcium stearate, talc, hydrogenated caster oil, sucrose esters of fatty acid, microcrystalline wax, yellow beeswax and white beeswax. The tablets may further comprise a coating.

The extended release tablet may release between 80% and 100% of the one or more active pharmaceutical ingredients over approximately eight hours in both an acidic environment of approximately 0.1N HCl and a near neutral environment of approximately pH 6.8.

In another general aspect there is provided a process for preparing extended release matrix tablets that include one or more water swellable cellulose derivatives, one or more alginic acid derivatives and one or more cationic polymers. The process includes dry blending the one or more water swellable cellulose derivatives, the one or more alginic acid derivatives, and the one or more cationic polymers together to form a blend.

Embodiments of the process may include one or more of the following features or the features described above. For example, the blend may further include one or more active pharmaceutical ingredients and/or one or more diluents. The process may further include dry granulating the blend to form granules, and compressing the granules to form tablets. The process instead may further include wet granulating the blend to form wet granules, drying and sizing the wet granules, and compressing the granules to form tablets. The process instead may further include incorporating one or more active pharmaceutical ingredients into the blend in geometric progression, mixing with lubricant and glidants, and directly compressing into tablets.

The water swellable cellulose derivative may be one or more or hydroxypropyl methylcellulose, hydroxypropyl cellulose, methylcellulose, carboxy methylcellulose, hydroxy methylcellulose, and hydroxy ethylcellulose. The alginic acid derivative may be one or more of alginic acid and its physiologically acceptable salts. The cationic polymer may be a methacrylic acid derivative with a dimethylaminoethyl ammonium group.

The tablets that result from the process may release between 80% and 100% of the active pharmaceutical ingredient in the extended release matrix tablet over approximately eight hours in both an acidic environment of approximately 0.1N HCl and a near neutral environment of approximately pH 6.8.

In another general aspect there is provided a method of treating a medical condition in need of pharmaceutical treatment. The method includes orally administering an extended release matrix tablet that includes one or more water swellable cellulose derivatives, one or more alginic acid derivatives and one or more cationic polymers, and one or more pharmaceutically active ingredients suitable for treatment of the medical condition for which the tablet is orally administered.

Embodiments of the method may include one or more of the following features or the features described above. For example, the water swellable cellulose derivative may be one or more of hydroxypropyl methylcellulose, hydroxypropyl cellulose, methylcellulose, carboxy methylcellulose, hydroxy methylcellulose, and hydroxy ethylcellulose. The alginic acid derivative may be one or more of alginic acid and its physiologically acceptable salts. The cationic polymer may be a methacrylic acid derivative with a dimethylaminoethyl ammonium group.

Between 80% and 100% of the active pharmaceutical ingredient in the extended release tablet is released over approximately eight hours in both an acidic environment of approximately 0.1N HCl and a near neutral environment of approximately pH 6.8.

The medical condition may be one or more conditions for which one or more of an antibiotic agent, a sympathomimetic agent, a sympatholytic agent, a cholinergic agent, an antimuscarinic agent, a gastro-intestinal drug, a gentio-urinary smooth muscle relaxant agent, a cardiac drug, an anticonvulsant agent, a tranquilizering agent and a sedative are suitable.

In another general aspect there is provided an extended release matrix tablet for oral administration that includes one or more active pharmaceutical ingredients and an extended release matrix. The extended release matrix includes between about 10% to about 50% by weight of the total formulation of a water swellable cellulose derivative, between 0.1% to about 15% by weight of the total formulation of an alginic acid derivative, and between 0.1% to about 15% by weight of the total formulation of a methacrylic acid derivative with a dimethylaminoethyl ammonium group. The active ingredient may be one or more of antibiotic agents, sympathomimetic agents, sympatholytic agents, cholinergic agents, antimuscarnics, gastro-intestinal drugs, gentio-urinary smooth muscle relaxants, cardiac drugs, anticonvulsant agents, tranquilizers and sedatives. Between 80% and 100% of the active pharmaceutical ingredient in the extended release tablet is released over approximately eight hours in both an acidic environment of approximately 0.1N HCl and a near neutral environment of approximately pH 6.8.

The details of one or more embodiments of the inventions are set forth in the description below. Other features, objects, and advantages of the invention will be apparent from the description and claims.

### DETAILED DESCRIPTION OF THE INVENTION

There is a pH gradient along the gastrointestinal tract that varies between the acidity of the stomach, the weakly acidic environment of the duodenum, and the neutral environment of the small intestine. In addition to this general variation in pH, there are fluctuations in pH arising from dietary changes. For example, fed and fasting states both affect the acidic environment of the stomach, and likewise would affect a drug product with a pH-dependent drug release if the drug were taken with or between meals. Extended release products providing pH independent drug release avoid bioavailability variations occurring due to these fluctuations of gastrointestinal pH. Therefore, it is desirable to achieve an extended release rate of a drug which is independent of pH and gastric residence time. Accordingly, there is provided an extended release matrix tablet that includes a water swellable cellulose derivative, an alginic acid derivative, and a cationic polymer, from which an active ingredient is released at a controlled rate.

The use of this polymer combination provides a desirable extended release matrix for oral administration from which active ingredient is released independent of pH and gastric residence time. In the acidic environment of the stomach the cellulose polymer absorbs water and swells to form a viscous consistency, which thereby retards the release of the drug. On the other hand the cationic polymer dissolves at the lower pH conditions causing the erosion of matrix, which exposes more drug to the dissolution media and consequently enhances the release rate. In the lower regions of the gastrointestinal tract as the pH rises, the solubility of cationic polymer decreases and it starts swelling whereas the alginic acid derivatives start dissolving causing erosion of the matrix. In this way, the present delivery system maintains a uniform rate of drug release independent of pH and gastric residence time throughout the gastrointestinal tract.

The term “pH independent release” as used herein refers to similar drug release rates varying not more than 20% when compared in acidic (0.1N HCl) and near neutral (pH 6.8) environments.

The extended release matrix tablet can be used for drugs independent of their solubility characteristics. Preferred active ingredients may be selected from one or more of antibiotics, sympathomimetics, sympatholytic agents, cholinergic agents, antimuscarinics, gastro-intestinal drugs, gentio-urinary smooth muscle relaxants, cardiac drugs, anticonvulsants, tranquilizers, and sedatives.

The water swellable cellulose derivatives that are used in the extended release tablet may be selected from one or more of hydroxypropyl methylcellulose, hydroxypropylcellulose, methylcellulose, carboxy methylcellulose, hydroxy methylcellulose, and hydroxy ethylcellulose. In particular, a suitable cellulose derivative is hydroxypropyl methylcellulose. Hydroxypropyl methylcellulose is commercially available as Methocel®, which is manufactured by Dow Chemicals and available in various grades. The preferred grades of Methocel® are K-4 MCR, K100V, K4MP, K15MP, K100MP, E4MP, E10MP-CR, ES. The water swellable cellulose derivative may constitute about 10% to about 50% by weight of the total weight of formulation.

The alginic acid derivatives that are used in the extended release tablets include both alginic acid and its physiologically acceptable salts such as those of sodium, potassium, magnesium and calcium. These compounds are commercially available in different grades. The preferred grades are Keltone LVCR and KELACID, which are marketed by ISP Alginates. The concentration of alginic acid derivatives may vary from about 0.1% to about 15% by weight of the total weight of formulation.

The cationic polymers that are used in the extended release tablets include methacrylic acid derivatives with a dimethylaminoethyl ammonium group. In particular, Eudragit® E100 and Eudragit® EPO, both of which are marketed by Rohm Pharma, may be selected. The weight of cationic polymer in the formulation may vary from about 0.1% to about 15% by weight with respect to the total weight of the formulation. According to the fourth addition of the Handbook of Pharmaceutical Excipients, Eudragit E is a cationic polymer based on dimethylaminoethyl methacrylate and other neutral methacrylic acid esters. It is soluble in gastric fluid as well as in weakly acidic buffer solutions (up to pH of approximately 5). The structure of Eudragit E is given in the handbook as: 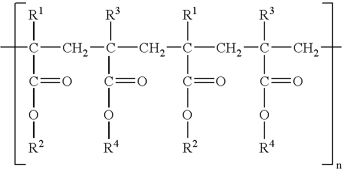
where: R¹=R³═CH₃
R¹=CH₂CH₂N(CH₃)₂
R¹=CH₃, C₄H₉

The dosage form may also contain other pharmaceutically inert excipients such as binders, diluents, lubricants, glidants and coloring agents. Suitable binders may be selected from one or more of methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate and propylene glycol. Suitable diluents may be selected from one or more of calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol starch, starch pregelatinized, sucrose, sugar compressible, sugar confectioners and mixtures thereof. Lubricants and glidants may be selected from one or more of colloidal anhydrous silica, stearic acid, magnesium stearate, calcium stearate, talc, hydrogenated caster oil, sucrose esters of fatty acid, microcrystalline wax, yellow beeswax and white beeswax. Suitable colors may be selected from any FDA approved colors for internal use. The formulation may optionally be coated, if desired.

The extended release matrix tablet may be prepared by blending the diluent and the control release polymers into a homogenous blend; incorporating the active drug ingredient into the blend in geometric progression; mixing with lubricant and glidant; and directly compressing into tablets. Alternatively, dry granulation or wet granulation methods can also be employed.

Mixing solid ingredients in a geometric progression generally refers to a process of adding almost equal amounts of two ingredients and then mixing to form a homogenous mixture of the two. This process is repeated by further mixing equal amounts to the mixture until the entire first ingredient is consumed. The entire mixture then is divided into, for example, four equal proportions and small amounts are taken from each portion and mixed thoroughly. This mixing is continued by adding from each portion until all the portions are completely used. The mixture then is further divided into two portions and the above process is repeated and ultimately the entire mixture is mixed randomly.

In one embodiment, a process for preparing extended release matrix tablets includes (a) dry blending the mixture of control release polymers and active drug ingredient into a homogeneous blend; (b) dry granulating the drug mixture from step (a); and (c) compressing the granules to form tablets.

In another embodiment, a process for preparing extended release matrix tablets includes (a) dry blending the mixture of control release polymers and active drug ingredient into a homogeneous blend; (b) wet granulating the dry mixture from step (a); (c) drying and sizing the wet granules from step (b); and (d) compressing the granules to form tablets.

In a further embodiment, a process for preparing extended release matrix tablets includes (a) dry blending the mixture of control release polymers and active drug ingredient into a homogeneous blend; and (b) directly compressing into tablets.

The following examples further exemplify the inventions and are not intended to limit the scope of the inventions.

### EXAMPLE 1

| | Quantity |
|---|---|
| CONTENTS | (mg/tablet) |
| Cefaclor | 540.9 |
| Lactose | 18.1 |
| Hydroxypropyl methylcellulose (medium viscosity) | 11 |
| Hydroxypropyl cellulose | 25 |
| Hydroxypropyl methylcellulose (low viscosity) | 152 |
| Sodium alginate | 35 |
| Eudragit ® EPO | 5 |
| Magnesium stearate | 6.5 |
| Talc | 4.0 |
| Colloidal anhydrous silica | 2.5 |

Process:
1. Lactose, hydroxypropyl methylcellulose, hydroxypropyl cellulose, sodium alginate and Eudragit® EPO were sieved through #BSS 44 and mixed in a double cone blender for 20 minutes.
2. Cefaclor was passed through sieve #BSS 44 and blended with the above mixture for 20 minutes.
3. The blend of step 3 was then mixed with talc and colloidal anhydrous silica for ten minutes.
4. The mixture of step 4 was lubricated by mixing with magnesium stearate for five minutes and compressed to form tablets.

### EXAMPLE 2

| | Quantity |
|---|---|
| CONTENTS | (mg/tablet) |
| Carvedilol | 50.90 |
| Lactose | 98.1 |
| Hydroxypropyl methylcellulose (medium viscosity) | 10 |
| Hydroxypropyl cellulose | 25 |
| Hydroxypropyl methylcellulose (low viscosity) | 100 |
| Sodium alginate | 5 |
| Alginic Acid | 10 |
| Eudragit ® EPO | 40 |
| Magnesium stearate | 3 |
| Talc | 2 |
| Colloidal anhydrous silica | 1 |

Process:
1. Lactose, hydroxypropyl methylcellulose, hydroxypropyl cellulose, sodium alginate, alginic acid and Eudragit® EPO were sieved through #BSS 44 and mixed in a double cone blender for 20 minutes.
2. Carvedilol was passed through sieve #BSS 44 and blended with the above mixture for 20 minutes.
3. The blend of step 3 was mixed with talc and colloidal anhydrous silica for ten minutes.
4. The mixture of step 4 was lubricated by mixing with magnesium stearate for five minutes and compressed to form tablets.

### EXAMPLE 3

| | Quantity |
|---|---|
| CONTENTS | (mg/tablet) |
| Carvedilol | 50.16 |
| Lactose | 99.84 |
| Hydroxypropyl methylcellulose (medium viscosity) | 35 |
| Hydroxypropyl cellulose | 25 |
| Hydroxypropyl methylcellulose (low viscosity) | 97 |
| Sodium alginate | 7 |
| Alginic acid | 10 |
| Eudragit ® EPO | 20 |
| Magnesium stearate | 3 |
| Talc | 2 |
| Colloidal anhydrous silica | 1 |

Process:
1. Carvedilol, lactose and hydroxypropyl methylcellulose (low viscosity) were sieved by passing through #BSS 44 and blended.
2. The blend was granulated by mixing with water followed by drying at 60° C. and sizing through sieve #BSS 30.
3. Hydroxypropyl cellulose, hydroxypropyl methylcellulose, alginic acid derivatives and Eudragit® EPO were passed through sieve #BSS 44 and blended in double cone blender for ten minutes.
4. The granules of step 2 were then mixed with the blend of step 3 for 20 minutes.
5. Talc and colloidal anhydrous silica were passed through # BSS44 and mixed with the blend of step 4 for five minutes.
6. The mixture of step 5 was finally lubricated by mixing with magnesium stearate (passed through #BSS44) for five minutes and compressed to form tablets.

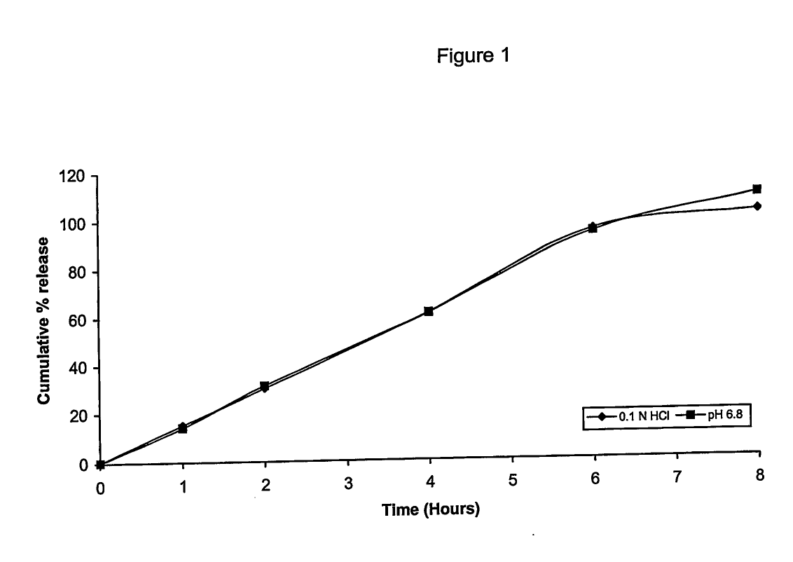

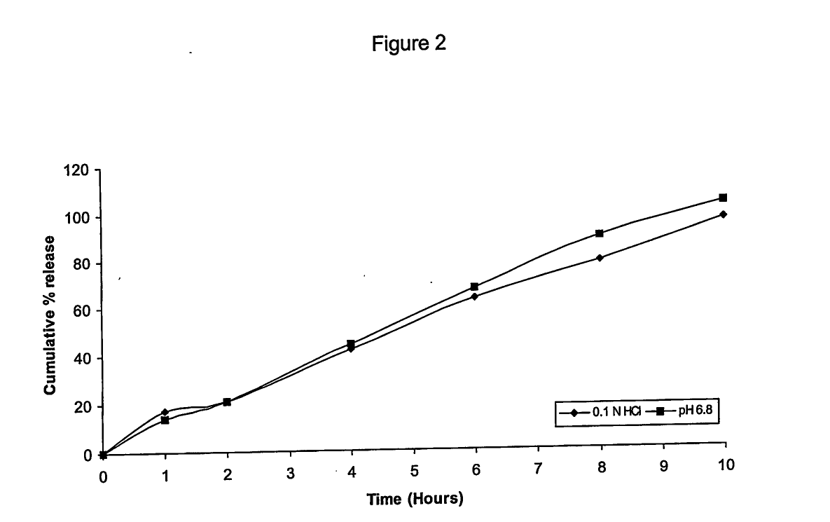

FIGS. 1 and 2 represent the in vitro release profiles of Carvedilol from the tablets prepared as per the compositions and processes of Example 2 and 3 respectively, in both acidic (0.1N HCl) and near neutral (Tri-sodium orthophosphate buffer with 1% sodium lauryl sulfate, pH 6.8) environments. The overlapping nature of the profiles clearly indicates the efficacy of the delivery system in maintaining similar release rates independent of pH.

While several particular forms of the invention have been described, it will be apparent that various modifications and combinations of the invention detailed in the text can be made without departing from the spirit and scope of the invention. Further, it is contemplated that any single feature or any combination of optional features of the inventive variations described herein may be specifically excluded from the claimed invention and be so described as a negative limitation. Accordingly, it is not intended that the invention be limited, except as by the appended claims.

1. An extended release matrix tablet for oral administration comprising one or more active pharmaceutical ingredients, a water swellable cellulose derivative, an alginic acid derivative and a cationic polymer.

2. The extended release matrix tablet according to claim 1, wherein the water swellable cellulose derivative comprises one or more or hydroxypropyl methylcellulose, hydroxypropyl cellulose, methylcellulose, carboxy methylcellulose, hydroxy methylcellulose, and hydroxy ethylcellulose.

3. The extended release matrix tablet according to claim 2, wherein the water swellable cellulose derivative comprises hydroxypropyl methylcellulose.

4. The extended release matrix tablet according to claim 2, wherein the water swellable cellulose derivative comprises hydroxypropyl cellulose.

5. The extended release matrix tablet according to claim 1, wherein the alginic acid derivative comprises one or more of alginic acid and its physiologically acceptable salts.

6. The extended release matrix tablet according to claim 5, wherein the physiologically acceptable alginic acid salts comprise one or more of sodium, potassium, calcium and magnesium salts of alginic acid.

7. The extended release matrix tablet according to claim 6, wherein the physiologically acceptable alginic acid salt comprises sodium alginate.

8. The extended release matrix tablet according to claim 1, wherein the cationic polymer comprises a methacrylic acid derivative with a dimethylaminoethyl ammonium group.

9.-10. (canceled)

11. The extended release matrix tablet according to claim 1, wherein the extended release matrix comprises from about 10% to about 80% by weight of the total formulation.

12. The extended release matrix tablet according to claim 1, wherein the water swellable cellulose polymer comprises from about 10% to about 50% by weight of the total formulation.

13. The extended release matrix tablet according to claim 1, wherein the alginic acid derivative comprises from about 0.1% to about 15% by weight of the total formulation.

14. The extended release matrix tablet according to claim 1, wherein the cationic polymer comprises from about 0.1% to about 15% by weight of the total formulation.

15. The extended release matrix tablet according to claim 1, wherein the active pharmaceutical ingredient comprises one or more of antibiotics, sympathomimetics, sympatholytic agents, cholinergic agents, antimuscarinics, gastro-intestinal drugs, gentio-urinary smooth muscle relaxants, cardiac drugs, anticonvulsants, tranquilizers and sedatives.

16. The extended release matrix tablet according to claim 15, wherein the active pharmaceutical ingredient comprises an antibiotic.

17. The extended release matrix tablet according to claim 16, wherein the antibiotic comprises cefaclor.

18. The extended release matrix tablet according to claim 15, wherein the active pharmaceutical ingredient comprises a sympatholytic agent.

19. The extended release matrix tablet according to claim 18, wherein the sympatholytic agent comprises carvedilol.

20.-25. (canceled)

26. A process for preparing extended release matrix tablets comprising one or more water swellable cellulose derivatives, one or more alginic acid derivatives and one or more cationic polymers, the process comprising:
dry blending the one or more water swellable cellulose derivatives, the one or more alginic acid derivatives, and the one or more cationic polymers together to form a blend.

27. The process of claim 27, wherein the blend further comprises one or more active pharmaceutical ingredients.

28. The process of claim 28, further comprising:
dry granulating the blend to form granules; and
compressing the granules to form tablets.

29. The process of claim 28, further comprising:
wet granulating the blend to form wet granules;
drying and sizing the wet granules; and
compressing the granules to form tablets.

30. The process of claim 27, wherein the blend further comprises one or more diluents.

31.-35. (canceled)

36. The process of claim 29, wherein between 80% and 100% of the active pharmaceutical ingredient in the extended release matrix tablet is released over approximately eight hours in both an acidic environment of approximately 0.1N HCl and a near neutral environment of approximately pH 6.8.

37. A method of treating a medical condition in need of pharmaceutical treatment, the method comprising orally administering an extended release matrix tablet comprising:
one or more water swellable cellulose derivatives, one or more alginic acid derivatives and one or more cationic polymers; and
one or more pharmaceutically active ingredients suitable for treatment of the medical condition for which the tablet is orally administered.

38.-42. (canceled)

43. An extended release matrix tablet for oral administration comprising one or more active pharmaceutical ingredients and an extended release matrix, wherein the extended release matrix comprises between about 10% to about 50% by weight of the total formulation of a water swellable cellulose derivative, between 0.1% to about 15% by weight of the total formulation of an alginic acid derivative, and between 0.1% to about 15% by weight of the total formulation of a methacrylic acid derivative with a dimethylaminoethyl ammonium group;
the active ingredient comprises one or more of antibiotic agents, sympathomimetic agents, sympatholytic agents, cholinergic agents, antimuscarinics, gastro-intestinal drugs, gentio-urinary smooth muscle relaxants, cardiac drugs, anticonvulsant agents, tranquilizers and sedatives; and
between 80% and 100% of the active pharmaceutical ingredient in the extended release tablet is released over approximately eight hours in both an acidic environment of approximately 0.1N HCl and a near neutral environment of approximately pH 6.8.
